(19) United States
(12) Patent Application Publication
(10) Pub. No.: US 2010/0136610 A1
(43) Pub. Date: Jun. 3, 2010

(54) Methods And Microfluidic Devices For Single Cell Detection Of Escherichia Coli

(76) Inventors: Jeong-Yeol Yoon, Tucson, AZ (US); Jae-Young Song, Anyang City (KR)

Correspondence Address: Nguyen & Tarbet, 6039 E. Grant Rd., Tucson, AZ, 85712, US

(21) Appl. No.: 12/629,797

(22) Filed: Dec. 2, 2009

Related U.S. Application Data
(60) Provisional application No. 61/200,702, filed on Dec. 3, 2008.

(51) Int. Cl. C12Q 1/04; C12M 1/34

(52) U.S. Cl. 435/34; 435/288.3

(57) ABSTRACT

The present invention features a microfluidic device for detecting Escherichia coli. The device comprises (a) a base slide having a first inlet and a second inlet, both of which connect at a vertex, where the first inlet is for accepting beads conjugated with anti-E. coli and the second inlet is for accepting a sample, wherein at the vertex the beads conjugated with anti-E. coli and the sample combine to form a combined mixture; (b) a portable spectrometer and a light source; and (c) a first fiber optic cable for directing an incident light into the combined mixture and a second fiber optic cable for detection of light scattering from the combined mixture, where the fiber optic cables are arranged in a proximity fiber arrangement, with the second fiber positioned above the base slide so as to detect forward light scattering at about a 45° angle.

### CROSS REFERENCE

This application claims priority to U.S. provisional application Ser. No. 61/200,702 filed Dec. 3, 2008, the specification of which is incorporated herein by reference in its entirety.

### FIELD OF THE INVENTION

The present invention is directed to a microfluidic device, more particularly to a microfluidic device and methods of use for detecting Escherichia coli.

### BACKGROUND OF THE INVENTION

Illnesses caused by waterborne pathogens range from mild gastrointestinal infections to life-threatening hemorrhagic colitis, haemolytic uremic syndrome, and thrombotic thrombocytopenic purpura. Accidental outbreaks of waterborne pathogens have recently increased in drinking and irrigation water; consequently, a growing interest in developing more effective methods for detecting waterborne pathogens has arisen. Conventional detection methods can be time-consuming due to sample preparation and the need for pre-culturing samples. This can make point-of-care and real-time detection very difficult. The present invention features a novel microfluidic device for detecting Escherichia coli. The present invention also features novel methods of detecting Escherichia coli.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY

The present invention features a microfluidic device for detecting Escherichia coll. The device comprises (a) a base slide having a first inlet and a second inlet, the first inlet and second inlet connect at a vertex, the first inlet is for accepting beads conjugated with anti-E. coli and the second inlet is for accepting a sample, wherein at the vertex the beads conjugated with anti-E. coli and the sample combine to form a combined mixture; (b) a portable spectrometer and a light source; and (c) a first fiber optic cable for directing an incident light into the combined mixture and a second fiber optic cable for detection of light scattering from the combined mixture, the fiber optic cables are arranged in a proximity fiber arrangement, the second fiber is positioned above the base slide so as to detect forward light scattering at about a 45° angle.

In some embodiments, the first inlet and the second inlet of the device have a width of about 200 μm. In some embodiments, the first inlet and the second inlet of the device have a depth of about 100 μm. In some embodiments, a view cell is constructed in the middle of a merged microchannel that has a much longer depth (e.g., 1 mm) than that of a channel (e.g., 100 μm) to help get a sufficient light path length. In some embodiments, the device further comprising a first glass slide bound on a top surface of the base slide and a second glass slide bound on a bottom surface of the base slide to enclose the microchannel. In some embodiments, the first inlet and the second inlet of the device connect via Teflon® tubes. In some embodiments, the device further comprising a syringe pump for injecting both the beads conjugated with anti-E. coli and the sample into the first inlet and the second inlet, respectively.

The present invention also features a method of detecting Escherichia coli. In some embodiments, the method comprises: (a) providing a microfluidic device comprising a base slide having a first inlet and a second inlet, both of which connect at a vertex; a portable spectrometer and a light source; and a first fiber optic cable for directing an incident light into the combined mixture and a second fiber optic cable for detection of light scattering from the combined mixture, where the fiber optic cables are arranged in a proximity fiber arrangement, with the second fiber positioned above the base slide so as to detect forward light scattering at about a 45° angle; (b) introducing beads conjugated with anti-E. coli to the first inlet and introducing a sample to the second inlet, the beads conjugated with anti-E. coli and the sample combine at the vertex to form the combined mixture; (c) subjecting the combined mixture to an incident light via the first fiber optic cable; and (d) detecting forward light scattering at a 45 degree angle via the second fiber optic cable.

In some embodiments, the method further comprises determining I₀ from the forward scattered light that is detected from the second sample and comparing I with I₀. Both I and I₀ are light intensities of forward light scattering, as measured by a portable spectrometer. Light scattering intensity (I) is a function of wavelength of an incident beam (λ), scattering angle (θ), refractive index of beads (n) and diameter of beads (d). Both I and I₀ varies upon integration time and the spectrometer used and have arbitrary unit (AU). In some embodiment, both I and I₀ have a range from 0 to 65535 (16-bit). In some embodiments, a difference between I and I₀ is calculated by subtracting of I₀ from of I. In some embodiments, a difference of greater than 0 indicates the presence of the microorganism in the sample.

### BRIEF DESCRIPTION OF THE DRAWINGS

FIG. 1A is a two-well slide and a Y-shape microfluidic device with the schematic illustration for the experimental procedure.

FIG. 1B is a side view of the slide of a microfluidic device.

FIG. 1C is a microfluidic device and proximity optical fibers with a portable spectrometer and a UV (380 nm) light source, for optical fiber detection.

FIG. 2 shows fluorescent microscopic images of stained E. coli cells in phosphate buffered saline (PBS) without washing (top) and with washing (bottom).

FIG. 3 shows light scattering intensities of immunoagglutinated E. coli K-12 in phosphate buffered saline (PBS) at various dilutions (10⁻⁵ to 10⁻⁸). Anti-E. coli were conjugated at 33% surface coverage to 0.02% (w/v), 0.92-μm highly carboxylated polystyrene particles (parking area=10.3 Å²). FIG. 3A shows results from a microfluidic device immunoassay. FIG. 3B shows results from a two-well slide immunoassay. All data are the intensity difference of scattered light with and without analyte. Error bars are standard deviation. * represents significant difference from blank signal.

### DESCRIPTION OF PREFERRED EMBODIMENTS

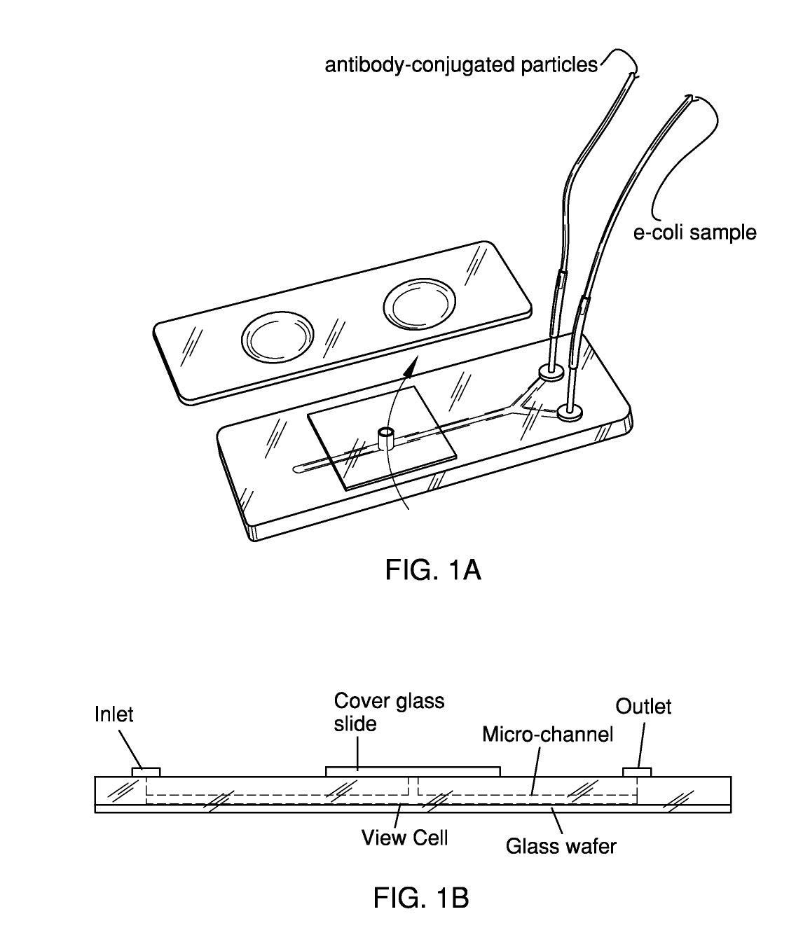

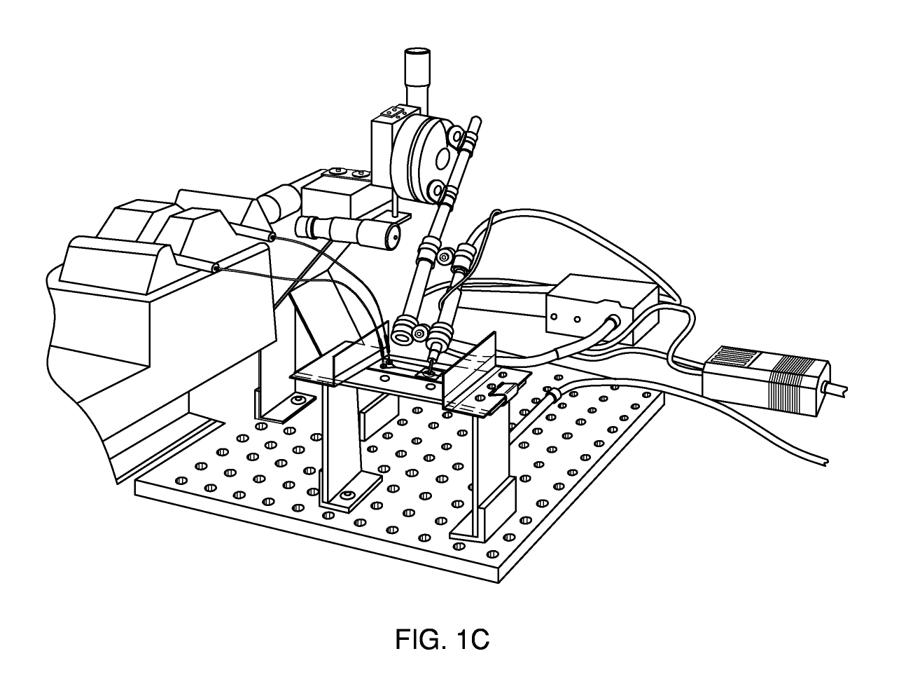

Referring now to FIG. 1-3, the present invention features a novel microfluidic device for detecting E. coli and novel methods of detecting E. coli. The microfluidic device of the present invention utilizes “proximity” optical fibers (e.g., the fibers are in close contact but not touching the microfluidic device) to quantify increased light scattering due to latex immunoagglutination in a microfluidic device. In some embodiments, highly carboxylated submicron particles with no surfactant are used.

### Conjugation of an Antibody

One (1) ml of 0.02% (w/v) 0.92-μm highly carboxylated polystyrene (HCPS) particles (10.3 Å² parking area per carboxyl surface group Bangs Laboratories, Fishers, Ind.) can be conjugated with 1 ml of 1.023 μg/ml anti-E. coli (e.g., polyclonal antibody developed in rabbit; catalog number ab13626; Abcam, Cambridge, Mass.) via physical adsorption. Surface coverage of antibodies to particles may be about 33%.

Culturing of Escherichia coli

E. coli K-12 lyophilized cell powder (Sigma-Aldrich catalog number EC1) can be cultured in media, for example brain heart infusion broth (Remel, Lenexa, Kans.), at about 37° C. for about 20 h. The grown cell culture of lyophilized E. coli K-12 can be serially diluted with 10 mM PBS (pH 7.4) by 10⁻⁵ to 10⁻⁸. As the lyophilized powder of E. coli K-12 may contain dead cell fragments and free antigen, the diluted E. coli K-12 solutions can be washed by centrifuging at about 2000 g for about 15 min, followed by elimination of supernatants and resuspension in PBS. This centrifugation-resuspension can be repeated (e.g., 3 times) to help ensure complete removal of dead cell fragments and free antigens.

For comparison with light scattering readings, a viable cell count can be performed by planting dilutions (e.g., abut 200 μl) to eosin methylene blue agar (DIFCO, Lawrence, Kans.) and incubating at about 37° C. for about 20 h. To stain viable and non-viable cells, SYTO 9 and propidium iodide (LIVE/DEAD BacLight viability kit; Invitrogen, Carlsbad, Calif.) can be used following the protocol as described in manufacturer's product information (Molecular Probes, 2004). Stained E. coli cells can be observed with a fluorescent microscope (Nikon, Tokyo, Japan). Cells can be counted using a Petroff-Hausser counting chamber (Electron Microscopy Sciences, Hatifield, Pa.).

### Fabrication of a Microfluidic Device

Microfluidic devices can be fabricated via standard soft lithography with a polydimethyl siloxane (PDMS) molding technique (well known to one of ordinary skill in the art). An example of a layout of a Y-shaped microfluidic device is shown in FIGS. 1A and 1B. The microfluidic device may comprise a slide (e.g., PDMS slide) with a first inlet (e.g., well) and a second inlet (e.g., well). The inlets (e.g., first inlet/well, second inlet/well) may be constructed to have a dimension of about 200 μm (width)×100 μm (depth) as measured by a profilometer (Alpha Step 2000, Tencor Instruments, Reston, Va.). In some embodiments, the inlets/wells may be constructed to have other dimensions.

In some embodiments, a second slide (e.g., PDMS slide) can be used as a cover in order to get a sufficient light path length (800 μm) in the view cell; however, this in some cases may make it difficult to acquire strong light scattering signals. In some embodiments, a hole can be made (e.g., diameter of about 2 mm; depth of about 2 mm) through the PDMS channel (e.g., using a hole puncher) to produce a view cell. Glass slides (e.g., the second slide, a third slide) can be bound on both top and bottom sides of the view cell, for example using oxygen plasma asher (Plasma Preen Cleaner/Etcher; Terra Universal, Fullerton, Calif.) at about 550 W for about 20 s (see FIG. 1B). The plasma bonding procedure can also make the PDMS hydrophilic, which can remain hydrophilic from about 24 h to about one week. This layout can produce a sufficient light path length, which may enhance the signal. The two inlets and one outlet can be then connected via Teflon® tubes (e.g., 0.79 mm OD; Upchurch Scientific, Oak Harbor, Wash.).

### Detection of Light Scattering

FIGS. 1A, 1B, and 1C show examples of an experimental setup for detecting light scattering using a microfluidic device according to the present invention. The setup comprises a portable spectrometer (e.g., a USB4000 miniature spectrometer), a light source (e.g., a model LS LED light source), and fiber optic cables (Ocean Optics, Dunedin, Fla.). The setup can be arranged in what is known as “proximity” fiber arrangement, for example the fiber distal ends are both very close (e.g., 1 mm) but not touching the microfluidic device. The two optical fibers for lighting and detection in the example have a 600 μm core diameter and 30 μm cladding with optimal transmission in the UV-visible wavelengths. The fibers are 1.0 meter in length with SMA-905 connectors (probes) on each end. The numerical aperture of these optical fibers and probes is 0.22 with an acceptance angle of about 25°. The 380 nm wavelength UV LED supplies about 45 μW power to the optical fiber assembly. The second fiber is positioned as a detector above the chip at about a 45° angle to measure light scattering while avoiding any of the direct incident light beam.

A syringe pump (KD Scientific, Holliston, Mass.) can be used to inject beads (e.g., microparticles) conjugated with anti-E. coli and samples (e.g., E. coli target solutions) to the Y-junction microchannel. Two Teflon® tubes (0.79 mm OD) can connect two 250-μl gastight syringes (Hamilton, Reno, Nev.) to the top openings of the PDMS substrate.

In some embodiments, two-well glass slides (model 48333, VWR, West Chester, Pa.) can be used (see FIG. 1A). These slides have two polished spherical depressions of about 18 mm diameter and about 800 μm depth. These may potentially lead to stronger signal.

Viable vs. Non-Viable E. coli Cells

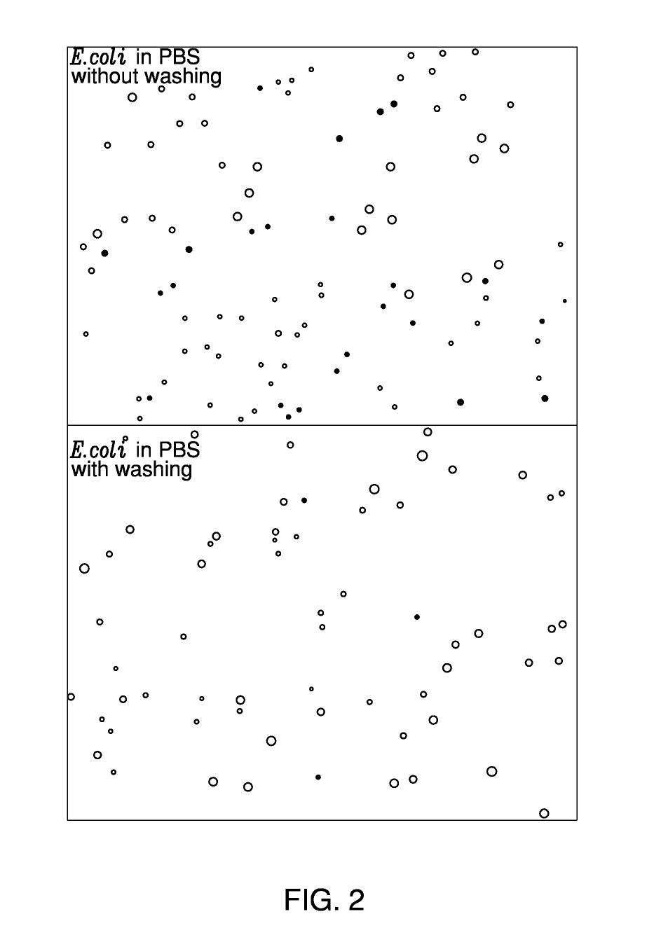

FIG. 2 shows the fluorescent microscopic images of stained E. coli in PBS buffer at a 10⁻² dilution, with or without washing (to remove dead cell fragments and free antigens). E. coli in PBS without washing showed the viable to non-viable ratio of approximately 4:1 (2.62×10⁷ viable cells/ml; 6.84×10⁶ non-viable cells/ml) as shown in FIG. 2 (left). Non-viable cell counts do not account for free antigens, because the fluorescent dyes (SYTO 9 and propidium iodide) in the LIVE/DEAD BacLight Bacterial Viability Kit stain nucleic acids (DNA and RNA). The number of free antigens that can be recognized by anti-E. coli would be substantially higher than the non-viable cell counts. The E. coli in PBS with washing showed a ratio of 100:1 (1.71×10⁷ viable cells ml-1; 1.71×10⁵ non-viable cells ml-1), showing E. coli cells are mostly viable (FIG. 2, right). The three times washing procedure enables the number of viable cells to be maintained while eliminating almost all non-viable cells.

Detection of E. coli Using Proximity Optical Fibers

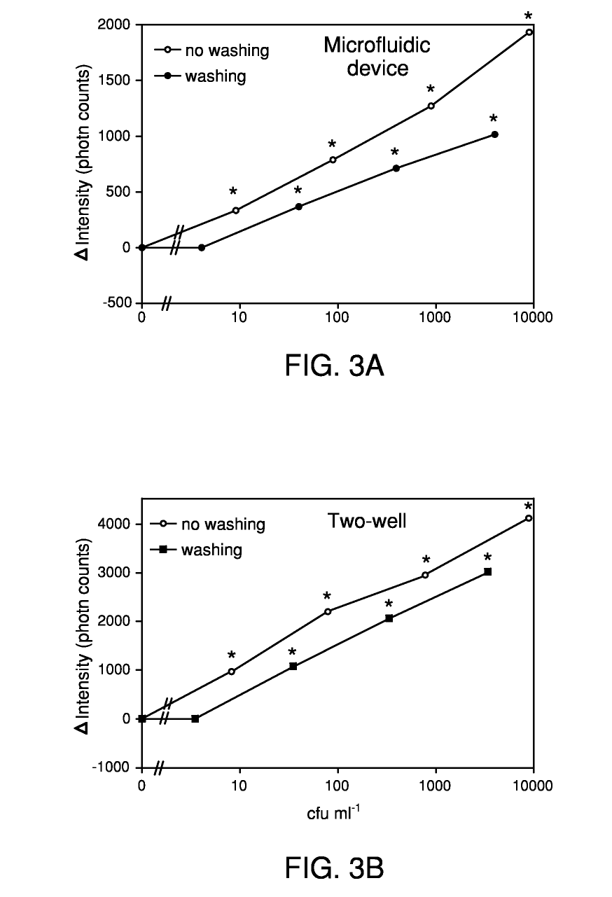

FIG. 3 shows the light scattering signals for E. coli K-12 in PBS, with or without washing, in two different setups; namely, two-well glass slide or microfluidic device. A total of four different dilutions were made: 10⁻⁵, 10⁻⁶, 10⁻⁷, and 10⁻⁸, thus making standard curves. PBS buffer was used as a negative control (blank). The presented light intensity signals in the standard curves were subtracted by blank signal, which includes no analyte. The data is comprised of the averages of five different experiments. The detection limit was determined by performing t-tests between the blanks and each dilution. The results in FIG. 3 indicate a significant difference between each dilution and the blank (p<0.05). The detection limit for E. coli in PBS buffer without washing was 9.1 cfu/ml. This detection limit is equivalent to <1 cfu per device considering the control volume (0.1 ml) of a microfluidic device. This remarkable sensitivity level may be overestimated, as we know from section 3.1 that there may exist a considerable number of dead E. coli without washing, subsequently releasing even more free antigens. These dead cells and free antigens also bind to anti-E. coli causing agglutination and increasing light scattering signal while not contributing to the number of colonies represented in cfu/ml units. The filled symbols in FIG. 3 show the results with washing, e.g., three times centrifuging and resuspending the E. coli culture, which eliminated dead cells and free antigens. This time, detection limit was 40 cfu/ml or 4 cfu per device. Although this detection limit is higher than those without washing, this level of sensitivity is greatly superior to the other detections performed in a microfluidic device. Both standard curves for two-well slide and microfluidic device showed linearity with changing concentration of E. coli, although the light intensity using two-well slide was stronger than that using microfluidic device. Through these calibration curves, partially quantification of specific concentration of E. coli K-12 can be available in the range of 10⁰-10⁴ cfu/ml.

The present invention features methods and microfluidic devices for real-time detection of E. coli through latex immunoagglutination. The microfluidic device utilizes proximity optical fibers. The methods are generally one-step and generally require no sample pre-treatment or cell culturing. The detection limit can be (but not limited to) as low as 40 cfu/ml or 4 cfu per device (viable cells only), or <10 cfu/ml or <1 cfu per device (including dead cells and free antigens).

As used herein, the term “about” refers to plus or minus 10% of the referenced number. For example, an embodiment wherein the detection limit is 10 cfu per ml includes a detection limit of between 9 and 11 cfu per ml.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

What is claimed is:

1. A microfluidic device for detecting Escherichia coli, said device comprising:
(a) a base slide having a first inlet and a second inlet, the first inlet and second inlet connect at a vertex, the first inlet is for accepting beads conjugated with anti-E. coli and the second inlet is for accepting a sample, wherein at the vertex the beads conjugated with anti-E. coli and the sample combine to form a combined mixture;
(b) a portable spectrometer and a light source; and
(c) a first fiber optic cable for directing an incident light into the combined mixture and a second fiber optic cable for detection of light scattering from the combined mixture, the fiber optic cables are arranged in a proximity fiber arrangement, the second fiber is positioned above the base slide so as to detect forward light scattering at about a 45° angle.

2. The microfluidic device of claim 1, wherein the first inlet and the second inlet have a width of about 200 μm.

3. The microfluidic device of claim 1, wherein the first inlet and the second inlet have a depth of about 100 μm.

4. The microfluidic device of claim 1 further comprising a view cell in the middle of a merged microchannel to help get a sufficient light path length.

5. The microfluidic device of claim 1 further comprising a first glass slide bound on a top surface of the base slide and a second glass slide bound on a bottom surface of the base slide.

6. The microfluidic device of claim 1, wherein the first inlet and the second inlet connect via Teflon® tubes.

7. The microfluidic device of claim 1 further comprising a syringe pump for injecting either the beads conjugated with anti-E. coli or the sample into the first inlet or the second inlet, respectively.

8. A method of detecting Escherichia coli, the method comprises:
(a) providing a microfluidic device comprising a base slide having a first inlet and a second inlet, both of which connect at a vertex; a portable spectrometer and a light source; and a first fiber optic cable for directing an incident light into the combined mixture and a second fiber optic cable for detection of light scattering from the combined mixture, where the fiber optic cables are arranged in a proximity fiber arrangement with the second fiber positioned above the base slide so as to detect forward light scattering at about a 45° angle;
(b) introducing beads conjugated with anti-E. coli to the first inlet and introducing a sample to the second inlet, the beads conjugated with anti-E. coli and the sample combine at the vertex to form the combined mixture;
(c) subjecting the combined mixture to an incident light via the first fiber optic cable; and
(d) detecting forward light scattering at a 45 degree angle via the second fiber optic cable.
